(19) United States
(12) Patent Application Publication
(10) Pub. No.: US 2026/0363772 A1
(43) Pub. Date: Nov. 19, 2026

(54) COHERENCE TOMOGRAPHY BASED LASER FIBER RANGING

(71) Applicant: GYRUS ACMI, INC. D/B/A OLYMPUS SURGICAL TECHNOLOGIES AMERICA, Westborough, MA (US)

(72) Inventor: Charles A. Baker, Rogers, MN (US)

(21) Appl. No.: 19/515,075

(22) PCT Filed: Aug. 21, 2024
(86) PCT No.: PCT/US2024/043177

§ 371 Date: Feb. 27, 2026

Related U.S. Application Data
(60) Provisional application No. 63/580,161, filed on Sep. 1, 2023.

(51) Int. Cl. A61B 18/26; A61B 1/00; A61B 18/00; A61B 18/22; A61B 34/30

(52) U.S. Cl. CPC A61B 18/26; A61B 1/00045; A61B 34/30; A61B 2018/00511; A61B 2018/00577; A61B 2018/00642; A61B 2018/00904; A61B 2018/00982; A61B 2018/2205; A61B 2034/301

(57) ABSTRACT

Systems, devices, and methods for providing adjustable laser treatment of a target using fiber-to-target distance measurements are disclosed. An exemplary surgical laser system includes a laser system to generate and deliver laser pulses to an anatomical target via an optical fiber, a feedback analyzer circuit to receive a return laser signal in response to a chirped laser irradiating at the target, and a controller circuit. The feedback analyzer circuit can generate an optical coherence metric using the chirped laser and the return laser signal, and determine a fiber-to-target distance between a distal end of the optical fiber and the target. Based at least on the fiber-to-target distance, the controller circuit can adjust a position or an orientation of the distal end of the optical fiber, or adjust a surgical laser output setting for generating and delivering laser pulses to the target.

### PRIORITY CLAIM

This application claims the benefit of priority to U.S. Provisional Patent Application Ser. No. 63/580,161 filed Sep. 1, 2024, the contents of which are incorporated herein by reference.

### FIELD OF THE DISCLOSURE

The present document relates generally to endoscopic laser surgical systems, and more specifically relates to systems and methods for measuring a distance between a laser fiber and a treatment target, and optimizing laser treatment based on the measured distance.

### BACKGROUND

Endoscopes have been used in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices or biological matter collection devices for sampling or treating an anatomical region, and providing suction passageways for collecting fluids (e.g., saline or other preparations), among other procedures. Examples of such anatomical region can include gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Some endoscopes include a working channel through which an operator can perform suction, placement of diagnostic or therapeutic devices (e.g., a brush, a biopsy needle or forceps, a stent, a basket, or a balloon), or minimally invasive surgeries such as tissue sampling or removal of unwanted tissue (e.g., benign or malignant strictures) or foreign objects (e.g., calculi). Some endoscopes can be used with a laser or plasma system to deliver energy to an anatomical target (e.g., soft or hard tissue or calculi) to achieve desired treatment. For example, laser has been used in applications of tissue ablation, coagulation, vaporization, fragmentation, and lithotripsy to break down calculi in kidney, gallbladder, ureter, among other stone-forming regions, or to ablate large calculi into smaller fragments.

### SUMMARY

When a laser surgical system is used to treat various diseases and conditions, the distance between a distal end of a laser fiber (or a distal tip of a device such as an endoscope integrating the laser fiber) and the anatomical target to be treated (hereinafter referred to as “fiber-to-target” distance) is an important factor that may determine a successful procedure. In an example of tissue ablation, if the laser fiber is too close to the target, flashing can occur along with fiber degradation and tissue sticking. If the fiber is too far from the target, more energy will be needed to achieve a desired tissue therapeutic effect.

Conventionally, an ideal or optimal fiber-to-target distance is heavily relied upon an operator's (e.g., an endoscopist's) experience. Based on such “best guess” of an ideal fiber-to-target distance, the operator manually positions the laser fiber with respect to the treatment target to achieve the desired fiber-to-target distance. This approach puts a high demand on the operator's experience, and accordingly may introduce inter-operator or inter-institution variations, particularly in difficult cases where the treatment target has complicated structures, compositions, or shapes, or be situated at hard-to-access locations. Manual positioning of the laser fiber to accord to the desired fiber-to-target distance usually takes significant amount of time and effort, and may cause fatigue in the operator, especially when fiber repositioning is constantly required throughout the procedure to treat complicated targets. For at least these reasons, the present inventor has recognized an unmet need for apparatus and techniques to automate the process of measuring the fiber-to-target distance and adjusting positions of the laser fiber during a laser procedure.

The present document describes systems, devices, and methods for measuring a fiber-to-target distance between a laser fiber and a treatment target during a laser procedure, and using the fiber-to-target distance measurement to optimize laser treatment of the target. An exemplary surgical laser system comprises a laser system to generate and deliver laser pulses to a target in an anatomical environment of a patient via an optical fiber, a feedback analyzer circuit to receive a return laser signal from the target in response to a chirped laser emitted from the laser system irradiating at the target, and a controller circuit. The feedback analyzer circuit can use at least a portion of the chirped laser and the return laser signal to generate an optical coherence metric correlated to roundtrip laser travel time between a distal end of the optical fiber and the target, and determine a fiber-to-target distance between a distal end of the optical fiber and the target based on the optical coherence metric. Based at least on the fiber-to-target distance, the controller circuit can controllably adjust a position or an orientation of the distal end of the optical fiber, and adjust a surgical laser output setting for generating and delivering laser pulses to the target.

Example 1 is a surgical laser system, including: a laser system configured to generate and deliver laser pulses to a target in an anatomical environment of a patient via an optical fiber; and a controller circuit, including a feedback analyzer circuit configured to: in response to a chirped laser emitted from the surgical laser system irradiating at the target, receive a return laser signal from the target; generate an optical coherence metric using at least a portion of the chirped laser and the return laser signal, the optical coherence metric correlated to roundtrip laser travel time between a distal end of the optical fiber and the target; and determine a fiber-to-target distance between the distal end of the optical fiber and the target using the optical coherence metric, wherein the controller circuit is configured to generate a control signal to adjust a position or an orientation of the distal end of the optical fiber relative to the target based at least in part on the determined fiber-to-target distance.

In Example 2, the subject matter of Example 1 optionally includes the feedback analyzer circuit that can be configured to determine the fiber-to-target distance further based on a chirp rate of the chirped laser.

In Example 3, the subject matter of any one or more of Examples 1-2 optionally includes the controller circuit that can be configured to adjust a surgical laser output setting of the surgical laser system based at least in part on the determined fiber-to-target distance.

In Example 4, the subject matter of any one or more of Examples 1-3 optionally includes a light source configured to direct an electromagnetic radiation at the target, wherein the feedback analyzer circuit is configured to:

detect a reflected imaging signal from the target in response to the electromagnetic radiation at the target; determine a spectroscopic property of the target from the reflected imaging signal; and identify a target type or composition based at least in part on the determined spectroscopic property of the target.

In Example 5, the subject matter of Example 4 optionally includes the controller circuit that can be configured to generate the control signal to adjust the position or the orientation of the distal end of the optical fiber relative to the target further based on the identified target type or composition.

In Example 6, the subject matter of any one or more of Examples 4-5 optionally includes the controller circuit that can be configured to adjust a surgical laser output setting of the surgical laser system based at least in part on the identified target type or composition.

In Example 7, the subject matter of any one or more of Examples 4-6 optionally includes the optical fiber that can be configured to concurrently direct the laser pulses and the reflected imaging signal.

In Example 8, the subject matter of any one or more of Examples 1-7 optionally includes the controller circuit that can be configured to provide the control signal to a robotic device coupled to the optical fiber to robotically adjust the position or the orientation of distal end of the optical fiber relative to the target.

In Example 9, the subject matter of any one or more of Examples 1-8 optionally includes a user interface configured to present to a user the determined fiber-to-target distance and a recommendation to adjust the position or the orientation of the distal end of the optical fiber relative to the target.

In Example 10, the subject matter of any one or more of Examples 1-9 optionally includes the target that can be a tissue target, wherein the surgical laser system is configured to generate and deliver the laser pulses to treat the tissue target.

In Example 11, the subject matter of any one or more of Examples 1-10 optionally includes the target that can be a calculi target, wherein the surgical laser system is configured to generate and deliver the laser pulses to ablate or fragment the calculi target.

In Example 12, the subject matter of any one or more of Examples 1-11 optionally includes an endoscope including or coupled to the surgical laser system, the endoscope including a longitudinal passage for passing the optical fiber.

In Example 13, the subject matter of any one or more of Examples 1-12 optionally includes the feedback analyzer circuit that can be further configured to: identify one or more outlier measurements from a plurality of fiber-to-target distance measurements generated over time; filter the plurality of fiber-to-target distance measurements to exclude the identified one or more outlier measurements; and determine the fiber-to-target distance using the filtered plurality of fiber-to-target distance measurements.

In Example 14, the subject matter of Example 13 optionally includes the feedback analyzer circuit that can be configured to identify the one or more outlier measurements based on an average and a variance of the plurality of fiber-to-target distance measurements.

Example 15 is a method of feedback-control of a surgical laser system during a laser surgery in a patient, the method including steps of: directing a chirped laser through an optical fiber of the surgical laser system at a target in an anatomical environment of the patient and receiving a return laser signal from the target in response to the chirped laser irradiating at the target; generating an optical coherence metric using at least a portion of the chirped laser and the return laser signal, the optical coherence metric correlated to roundtrip laser travel time between a distal end of the optical fiber and the target; determining a fiber-to-target distance between the distal end of the optical fiber and the target using the optical coherence metric; and adjusting a position or an orientation of the distal end of the optical fiber relative to the target based at least in part on the determined fiber-to-target distance.

In Example 16, the subject matter of Example 15 optionally includes determining the fiber-to-target distance is further based on a chirp rate of the chirped laser.

In Example 17, the subject matter of any one or more of Examples 15-16 optionally include adjusting a surgical laser output setting of the surgical laser system based at least in part on the determined fiber-to-target distance.

In Example 18, the subject matter of any one or more of Examples 15-17 optionally includes directing an electromagnetic radiation at the target; in response to the electromagnetic radiation, receiving a reflected imaging signal from the target; determining a spectroscopic property of the target from the reflected imaging signal; and identifying a target type or composition based at least in part on the determined spectroscopic property of the target.

In Example 19, the subject matter of Example 18 optionally includes adjusting the position or the orientation of the distal end of the optical fiber relative to the target that can be further based on the identified target type or composition.

In Example 20, the subject matter of any one or more of Examples 18-19 optionally includes adjusting a surgical laser output setting of the surgical laser system based at least in part on the identified target type or composition.

In Example 21, the subject matter of any one or more of Examples 15-20 optionally includes the target that can be a tissue target or a calculi target.

In Example 22, the subject matter of any one or more of Examples 15-21 optionally includes providing a control signal to a robotic device coupled to the optical fiber to robotically adjust the position or the orientation of the distal end of the optical fiber relative to the target.

In Example 23, the subject matter of any one or more of Examples 15-22 optionally includes presenting on a user interface the determined fiber-to-target distance and a recommendation to adjust the position or the orientation of the distal end of the optical fiber relative to the target.

In Example 24, the subject matter of any one or more of Examples 15-23 optionally includes identifying one or more outlier measurements from a plurality of fiber-to-target distance measurements generated over time each based on respective optical coherence metrics; filtering the plurality of fiber-to-target distance measurements to exclude the identified one or more outlier measurements; and determining the fiber-to-target distance using the filtered plurality of fiber-to-target distance measurements.

In Example 25, the subject matter of Example 24 optionally includes identifying the one or more outlier measurements based on an average and a variance of the plurality of fiber-to-target distance measurements.

The systems, devices, and methods described herein may be used in various endoscopic laser surgical procedures to improve surgical success rate. The fiber-to-target distance measurement and automated fiber positioning as described herein may help reduce inter-operator variability inherent to subjective “best guess” of the fiber-to-target distance, and produce more consistent and predictable surgical outcome. The optical time of flight (ToF) method as applied to the automated fiber-to-target distance measurement can produce more accurate and precise distance measurements. Compared to conventional methods which are limited by inaccuracy at short range due to the short flight time of the pulses, the ToF method, such as the optical coherence tomography (OCT) or frequency modulated continuous wave (FMCW) method can eliminate or reduce timing jitter, and provide a higher accuracy especially in short-range distance measurement. In accordance with some embodiments as described herein, the fiber-to-target distance measurements may be filtered by excluding outlier measurements automatically identified using a statistical outlier detector, which can result in a refined fiber-to-target distance. Automatically measuring the fiber-to-target distance and controlling the fiber position may lead to less user fatigue and faster procedures. The autonomous control of laser fiber advancement or retraction provides more effective use of the laser system. The precise fiber-to-target distance measurement and automatic control of the laser fiber position may also help prevent laser flashing events, reduce the heating effect of fluid and tissue surrounding the target, and achieve more efficient laser energy use and overall cost saving.

The systems, devices, and techniques as described in accordance with various embodiments in this document, may be used in various endoscopy procedures involving laser treatment of tissue or other targets, including, for example, colonoscopy, anoscopy, arthroscopy, bronchoscopy, colonoscopy, colposcopy, cystoscopy, esophagoscopy, gastroscopy, laparoscopy, laryngoscopy, neuroendoscopy, proctoscopy, sigmoidoscopy, thoracoscopy etc.

This summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWING

Various embodiments are illustrated by way of example in the figures of the accompanying drawings. Such embodiments are demonstrative and not intended to be exhaustive or exclusive embodiments of the present subject matter.

FIG. 1 is a block diagram illustrating an example of a laser energy delivery system configured to provide laser treatment to an anatomical target.

FIG. 2 is a block diagram illustrating a laser surgical system to provide adjustable laser treatment of a target using feedback information including fiber-to-target distance measurements.

FIG. 3 illustrates an example of a feedback-controlled endoscopic laser surgical system with automatic fiber-to-target distance measurement and optical fiber position control.

FIGS. 4A-4B are graphs illustrating the working principle of using a frequency modulated continuous wave (FMCW) method for estimating the fiber-to-target distance.

FIGS. 5A-5B are graphs illustrating an example of how air bubbles interfere with the fiber-to-target distance measurement.

FIG. 6 illustrates an example of overestimate of the fiber-to-target distance due to air bubbles present in the light path of the laser pulses.

FIG. 7 is a flow chart illustrating an example method of providing feedback-control of a surgical laser system to provide adjustable laser treatment of a target using feedback including fiber-to-target distance measurements.

FIG. 8 is a block diagram illustrating an example machine upon which any one or more of the techniques (e.g., methodologies) discussed herein may perform.

### DETAILED DESCRIPTION

Described herein are systems, devices, and methods for providing adjustable laser treatment of a target using feedback including fiber-to-target distance measurements. An exemplary surgical laser system comprises a laser system to generate and deliver laser pulses to a target in an anatomical environment of a patient via an optical fiber, a feedback analyzer circuit to receive a return laser signal from the target in response to a chirped laser emitted from the laser system irradiating at the target, and a controller circuit. The feedback analyzer circuit can use at least a portion of the chirped laser and the return laser signal to generate an optical coherence metric correlated to roundtrip laser travel time between a distal end of the optical fiber and the target, and determine a fiber-to-target distance between a distal end of the optical fiber and the target based on the optical coherence metric. The controller circuit can controllably adjust a position or an orientation of the distal end of the optical fiber, and adjust a surgical laser output setting for generating and delivering laser pulses to the target, based at least on the fiber-to-target distance.

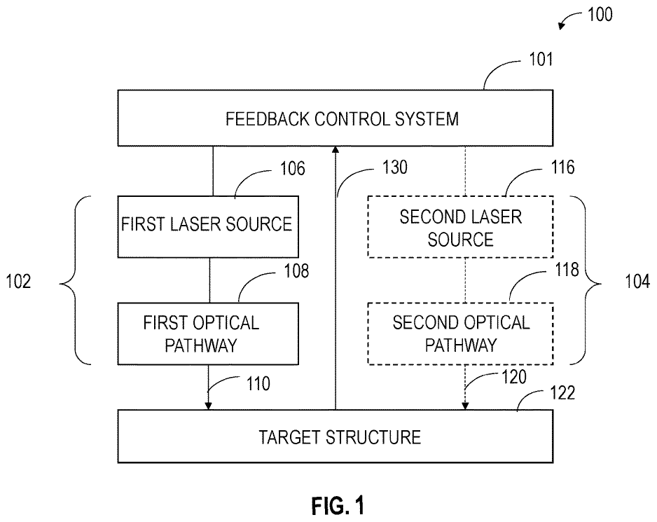

FIG. 1 is a block diagram illustrating an example of a laser energy delivery system 100 configured to provide laser treatment to a target structure 122 in an anatomical environment of a subject, such as anatomical structure (e.g., soft tissue, hard tissue, or abnormal such as cancerous tissue) or calculi structure (e.g., kidney or pancreobiliary or gallbladder stone). In some examples, the laser energy delivery system 100 may deliver precisely controlled therapeutic treatment of tissue or other anatomical structures (e.g., tissue ablation, coagulation, vaporization, or the like) or treatment of non-anatomical structures (e.g., ablation or dusting of calculi structures).

The laser energy delivery system 100 can include a feedback control system 101, and at least one laser system in operative communication with the feedback control system 101. By way of example and not limitation, FIG. 1 shows the laser feedback system connected to a first laser system 102 and optionally (shown in dotted lines) to a second laser system 104. Additional laser systems are contemplated within the scope of the present disclosure. The first laser system 102 may include a first laser source 106, and associated components such as power supply, display, cooling systems and the like. The first laser system 102 may also include a first optical pathway 108 operatively coupled with the first laser source 106. In an example, the first optical pathway 108 includes an optical fiber. The first optical pathway 108 may be configured to transmit laser beams from the first laser source 106 to the target structure 122.

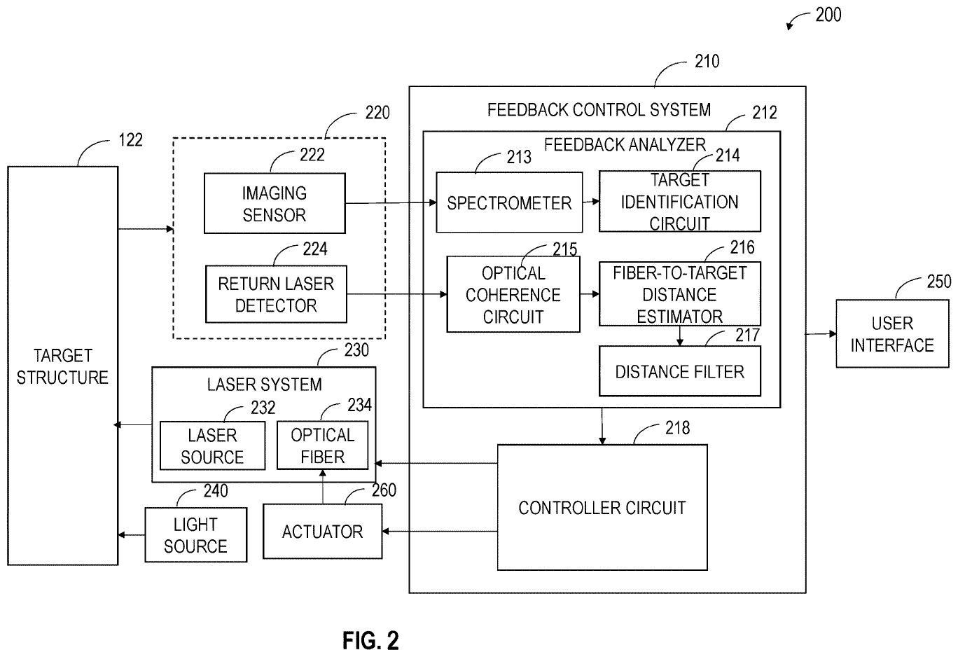

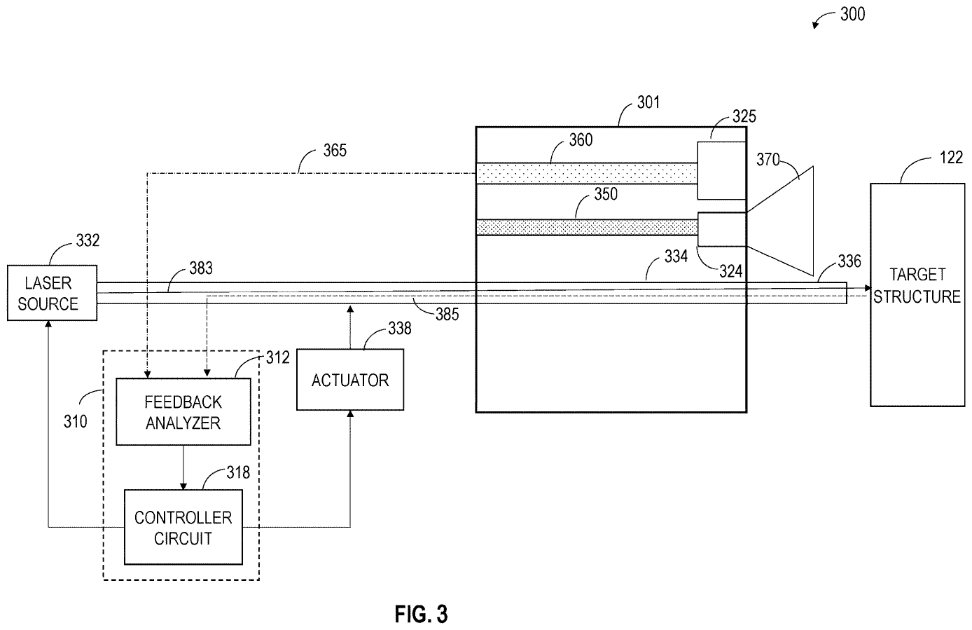

The feedback control system 101 may receive feedback signals 130 from the target. In an example, the feedback signals 130 may include signals indicative of target properties or surgical site conditions. In an example, the feedback signals 130 may include an acoustic signal produced by a laser pulse propagating through the media (e.g., liquid and vapor), projecting to the target and causing the target to vibrate. In another example, the feedback signals 130 may include reflected electromagnetic signal (e.g., reflected illumination light emitted from a light source). In an example, the feedback signals 130 may include images or video frames of at least a portion of the surgical site such as generated by an imaging sensor during a procedure. In yet another example, the feedback signals 130 may include a return laser signal in response to laser pulses irradiating at the target. The laser pulses may be generated by the first laser system 102 or the second laser system 104 in accordance with a specific output setting, such as a chirped laser. The return laser signal may be used for determining whether the target is within the laser firing range, as will be described further below with respect to FIGS. 2-3.

The feedback signals 130 may be used to control laser delivery, laser energy output, and/or other system parameters to improve therapy efficacy and to achieve or maintain a desired condition at the target site. In an example, the feedback control system 101 may analyze the feedback signals 130 to determine one or more target properties. Based on the determined target properties, the feedback control system 101 may identify target type or composition, adjust a laser output setting (e.g., one or more laser irradiation parameters such as power, duration, frequency, pulse shape, exposure time, or firing angle) or other system parameters, and generate and deliver laser pulses to the target in accordance with the laser output setting to achieve a desired therapeutic effect or to maintain a desired condition. For instance, the feedback control system 101 may monitor properties of the target structure during a therapeutic procedure (e.g., ablating calculi such as kidney stones into smaller fragments) to determine if the tissue was suitably ablated prior to another therapeutic procedure (e.g., coagulation of blood vessels). In another example, the feedback control system 101 may analyze the feedback signals 130 to automatically determine a distance between a distal end of the laser fiber and the target tissue to be treated, also referred to as a “fiber-to-target” distance in this document. The fiber-to-distance may be used to guide manual or autonomous positioning of the laser fiber (e.g., advancing or retracting, or changing an orientation of, the distal end of the laser fiber) to achieve more efficient laser treatment of the target.

In an example, the first laser source 106 may be configured to provide a first output 110. The first output 110 may extend over a first wavelength range, such as one that corresponds to a portion of the absorption spectrum of the target structure. The first output 110 may provide effective ablation and/or carbonation of the target structure since the first output 110 is over a wavelength range that corresponds to the absorption spectrum of the tissue.

In an example, the first laser source 106 may be configured such that the first output 110 emitted at the first wavelength range corresponds to high absorption (e.g., exceeding about 250 cm⁻¹) of the incident first output 110 by the tissue. In example aspects, the first laser source 106 may emit first output 110 between about 1900 nanometers (nm) and about 3000 nm (e.g., corresponding to high absorption by water) and/or between about 400 nm and about 520 nm (e.g., corresponding to high absorption by oxy-hemoglobin and/or deoxy-hemoglobin). Appreciably, there are two main mechanisms of light interaction with a tissue: absorption and scattering. When the absorption of a tissue is high (absorption coefficient exceeding 250 cm⁻¹) the first absorption mechanism dominates, and when the absorption is low (absorption coefficient less than 250 cm⁻¹), for example lasers at 800-1100 nm wavelength range, the scattering mechanism dominates.

Various commercially available medical-grade laser systems may be suitable for the first laser source 106. Examples of laser source 106 may include UV-VIS emitting In_{X}Ga_{1-X}N semiconductor lasers such as GaN laser with emission at 515-520 nm, In_{X}Ga_{1-X}N laser with emission at 370-493 nm, Ga_{X}Al_{1-X}As laser with emission at 750-850 nm, or In_{X}Ga_{1-X}As laser with emission at 904-1065 nm, among others. Alternatively, infrared (IR) lasers such as those summarized in Table 1 below may be used.

TABLE 1
Example List of suitable IR lasers

| | Wavelength | Absorption Coefficient | Optical Penetration Depth |
|---|---|---|---|
| Laser | λ (nm) | μ [symbol] (cm⁻¹) | δ (μm) |
| Thulium fiber laser: | 1908 | 88/150 | 114/67 |
| Thulium fiber laser: | 1940 | 120/135 | 83/75 |
| Thulium:YAG: | 2010 | 62/60 | 161/167 |
| Holmium:YAG: | 2120 | 24/24 | 417/417 |
| Erbium:YAG: | 2940 | 12,000/1,000 | 1/10 |

[symbol]

indicates data missing or illegible when filed

The optional second laser system 104 may include a second laser source 116 for providing a second output 120, and associated components, such as power supply, display, cooling systems and the like. The second laser system 104 may either be operatively separated from or, in the alternative, operatively coupled to the first laser source 106. In some embodiments, the second laser system 104 may include a second optical pathway 118 (separate from the first optical pathway 108) operatively coupled to the second laser source 116 for transmitting the second output 120. Alternatively, the first optical pathway 108 may be configured to transmit both the first output 110 and the second output 120.

In certain aspects, the second output 120 may extend over a second wavelength range, distinct from the first wavelength range. Accordingly, there may not be any overlap between the first wavelength range and the second wavelength range. Alternatively, the first wavelength range and the second wavelength range may have at least a partial overlap with each other. In advantageous aspects of the present disclosure, the second wavelength range may not correspond to portions of the absorption spectrum of the target structure where incident radiation is strongly absorbed by tissue that has not been previously ablated or carbonized. In some such aspects, the second output 120 may advantageously not ablate uncarbonized tissue. In another embodiment, the second output 120 may ablate carbonized tissue that has been previously ablated. In additional embodiments, the second output 120 may provide additional therapeutic effects. For instance, the second output 120 may be more suitable for coagulating tissue or blood vessels.

FIG. 2 is a block diagram illustrating a laser surgical system 200 to provide a feedback-controlled laser treatment of a target using feedback information including fiber-to-target distance measurements. The system 200 can be an embodiment of the laser energy delivery system 100 for treating anatomical targets of various types, or a lithotripsy system for destructing hardened masses like kidney stones, bezoars, gallstone, among other calculi structures.

The laser surgical system 200 can includes a feedback control system 210, one or more sensors 220, a laser system 230, a light source 240, a user interface 250, and an actuator 260. The laser system 230, which is an example of the laser system 102 or the laser system 104 shown in FIG. 1, can include a laser source 232 (which can be an example of the first laser source 106 or the second laser source 116) and an optical fiber 234 (which can be the first optical pathway 108 or the second optical pathway 118) for directing the laser energy to the target structure 122. The laser source 232 may generate laser pulses in accordance with an output setting, which may include one or more laser irradiation parameters (e.g., intensity, power, duration, frequency, or pulse shape, exposure time, or firing angle). At least some of the laser irradiation parameters are programmable or adjustable either automatically such as by the controller circuit 218, or manually by a user via the user interface 250. The laser pulses may be used for therapeutic purposes, such as for surgically removing or sampling tissue or ablating a calculi structure. In some examples, the laser source 232 may generate laser pulses for non-therapeutic uses, such as for estimating a fiber-to-target distance, as described in this document in accordance with various embodiments. In some examples, the laser source 232 may include distinct laser sources, including a first laser source (such as the first laser source 106 shown in FIG. 1) to generate the therapeutic laser pulses, and a second lase source (such as the second laser source 116 shown in FIG. 1) to generate non-therapeutic laser pulses that may be used for estimating a fiber-to-target distance.

The feedback control system 210, which is an embodiment of the feedback control system 101 shown in FIG. 1, can include a feedback analyzer 212 and a controller circuit 218. According to example embodiments, the feedback control system 210, or a part thereof (such as the feedback analyzer 212 and/or the controller circuit 218) may include processors, such as microprocessors, digital signal processors (DSPs), application specific integrated circuits (ASICs), field programmable gate arrays (FPGAs), or any other equivalent integrated or discrete logic circuitry, as well as any combinations of such components for performing one or more of the functions attributed to the feedback control system 210.

The feedback analyzer 212 may be communicatively coupled to one or more sensors 220, receive therefrom feedback information, determine a target property using the feedback information, and control the laser system 230 to provide appropriate laser treatment based at least in part on the target property. By way of example and not limitation and as illustrated in FIG. 2, the one or more sensors 220 may include an imaging sensor 222 and a return laser detector 224. The imaging sensor 222 can be included in an imaging system that further includes a lens system. Examples of the imaging sensor 222 can include a CCD or CMOS camera sensitive in ultraviolet (UV), visible (VIS) or infrared (IR) wavelengths. The imaging sensor 222 can be located a distal portion of an endoscope for use during the procedure, an example of which is illustrated in FIG. 3. The imaging sensor 222 may obtain an imaging signal of at least a portion of the target structure 122 during the procedure. In an example, the light source 240 can generate and direct electromagnetic radiation at the target structure 122, and the imaging sensor 222 can obtain the imaging signal in response to the electromagnetic radiation incident on the target structure 122. Table 2 below shows examples of the light source 240 as applicable to the examples discussed herein.

TABLE 2
Light sources for spectroscopic system

| Application | Wavelength Range | | Type |
|---|---|---|---|
| Color/VIS/NIR | 360-2500 | nm | Tungsten Halogen |
| DUV | 190-400 | nm | Deuterium |
| UV | 215-400 | nm | Deuterium |
| UV/VIS/NIR | 215-2500 | nm | Deuterium/Halogen |
| reflection/absorption | | | |
| UV/VIS/NIR | 200-2500 | nm | Deuterium/Halogen |
| absorption | | | |
| UV/VIS | 200-1000 | nm | Xenon |
| FTIR | 2000-25000 | nm | Silicon Carbide |

| | | |
|---|---|---|
| UV/VIS/IR | Multiple narrow | LED, Laser Diode |
| Fluorescence | emitting | |

The return laser detector 224 may detect a return laser signal from the target structure 122 in response to an excitation laser irradiating at the target structure 122. The excitation laser pulses can be emitted from the laser system 230. The return laser detector 224 may be positioned at the distal end of the optical fiber 234 in proximity of the fiber tip from which the excitation laser pulses are emitted. In an example, the excitation laser may include a chirped laser, also known as a frequency-swept laser, which has a time-varying instantaneous frequency. The laser system 230 can include an optical splitter that splits the chirped laser into a first portion directed to and irradiating at the target structure 122, and a second portion being kept local and does not travel to the target structure 122. The return laser signal from the target, in response to the first portion irradiating at the target, can be detected, and interferometrically recombined with the second portion of the chirped laser. As will be described further below, a coherence metric may be determined and used for estimating the fiber-to-target distance.

The imaging signal obtained by the imaging sensor 222 and the return laser signal detected by the return laser detector 224 may be provided to the feedback analyzer 212. The feedback analyzer 212 includes one or more of a spectrometer 213, a target identification circuit 214, an optical coherence circuit 215, a fiber-to-target distance estimator 216, and a distance filter 217. The spectrometer 213 may determine one or more spectroscopic properties from the imaging signal of the target, such as reflectivity, absorption index, among other spectral properties. Examples of the spectrometer 213 may include a Fourier Transform Infrared spectrometer (FTIR), a Raman spectrometer, a UV-VIS reflection spectrometer, a UV-VIS-IR spectrometer, a fluorescent spectrometer, and the like. The FTIR is a method used for routine, easy and rapid materials analysis. This technique has relatively good spatial resolution and gives information about the chemical composition of the material. The Raman spectroscopy has good accuracy in identifying hard and soft tissue components. As a high spatial resolution technique, it is also useful for determining distribution of components within a target. The UV-VIS reflection spectroscopy is a method that gathers information from the light reflected off an object similar to the information yielded from the eye or a color image made by a high-resolution camera, but more quantitatively and objectively. The reflection spectroscopy offers information about the material since light reflection and absorption depends on its chemical composition and surface properties. It is also possible to get unique information about both surface and bulk properties of the sample using this technique. The reflection spectroscopy can be a valuable technique to recognize composition of hard or soft tissue. The fluorescent spectroscopy a type of electromagnetic spectroscopy that analyzes fluorescence from a sample. It involves using a beam of light, usually ultraviolet, that excites a material compound and causes the material compound to emit light, typically in visible or IR area. The method is applicable for analysis of some organic components such as hard and soft tissue.

The target identification circuit 214 may identify in vivo a type or composition of the target structure 122 (or a specific portion thereof) during the procedure using the one or more spectroscopic properties determined by the spectrometer 213. In endoscopic laser therapy, it is desirable to identify target type and composition, apply appropriate laser energy only to treatment target (e.g., cancerous tissue, or a particular calculus type) while avoiding or reducing laser irradiation at non-treatment tissue (e.g., normal tissue). Conventional target identification generally requires collecting a sample of the target for in vitro analysis. Continuous monitoring and automatic in vivo tissue identification at the tip of the endoscope may advantageously reduce surgery time and complexity, give physicians more information to better adapt the treatment during the procedure, and improve therapy efficacy. For example, in laser lithotripsy that applies laser to break apart or dust calculi, automatic and in vivo recognition of calculi of a particular type (e.g., chemical composition of a kidney or pancreobiliary or gallbladder stone) and distinguishing it from surrounding tissue would allow a physician to adjust a laser setting (e.g., power, exposure time, or firing angle) to more effectively ablate the target stone, while at the same time avoiding irradiating neighboring non-treatment tissue. Commonly assigned U.S. patent application Ser. No. 16/947,488, entitled “LASER FIBER-TO-TARGET DISTANCE CONTROL,” describes example methods of identifying or classifying different target structures, such as different compositions of kidney stones (e.g., calcium oxalate stone (Monohydrate), calcium oxalate stone (Dihydrate), calcium phosphate stone, struvite stone, and uric acid stone) using spectroscopic data, the description of which is hereby incorporated by reference in its entirety.

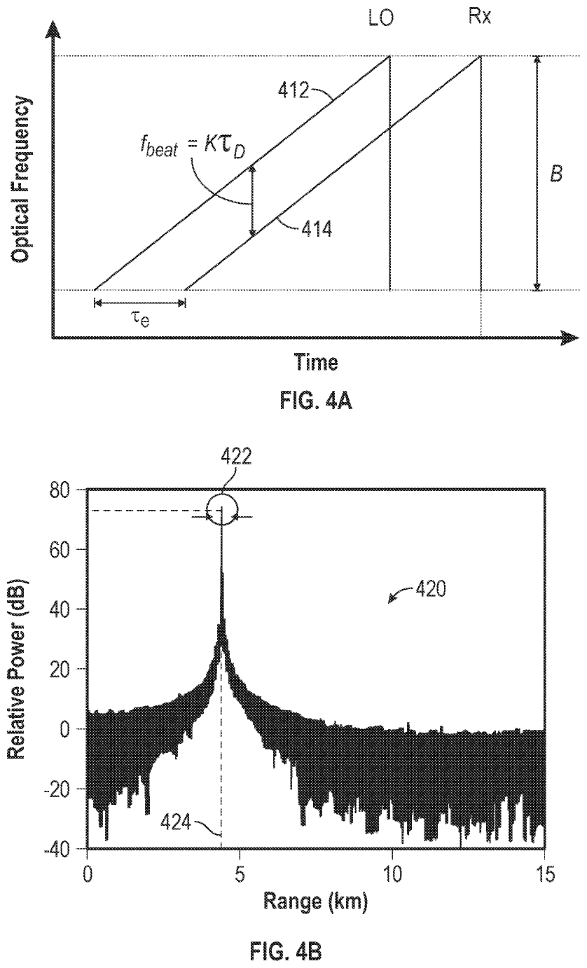

The optical coherence circuit 215 can compute a coherence metric using the return laser signal from the target interferometrically recombined with the second portion of the chirped laser split from the chirped laser pulses. The optical coherence metric may involve an interferogram and analysis of frequency components of the interferogram. The fiber-to-target distance estimator 216 may estimate a distance between a distal end of the optical fiber 234 and the target structure 122 (the “fiber-to-target distance”) based at least in part on the coherence metric. The optical coherence-based distance measurement method is also known as frequency modulated continuous wave (FMCW) method, which has been implemented in LiDAR (light detection and ranging) scanner and found vast object ranging applications in land management and planning, hazard assessment, forestry, agriculture, geologic mapping, or watershed and river surveys. FIGS. 4A-4B are graphs illustrating the working principle of FMCW method for measuring a fiber-to-target distance. FIG. 4A illustrates conceptually a time-frequency representation 412 of the second portion of the chirped laser (LO) split from the chirped laser emitted from the laser system 230, and a time-frequency representation 414 of the return laser signal (RX) in response to the first portion of the chirped laser irradiating at the target structure 122. By way of non-limiting example and as illustrated, the chirped laser LO is a linear chirp with an optical frequency (on the y-axis) being a linear ramp function of time (on the x-axis). The return laser signal RX also has a linear ramp frequency over time that closely resembles the chirped laser LO, and is detected after a time delay To after the chirped laser LO. When the return laser detector 224 is positioned at the distal end of the optical fiber 234 in proximity of the fiber tip from which the excitation laser pulses are emitted, the time delay, τ_{D}, corresponds to a roundtrip laser travel time between the target structure 122 and the detector 224. Specifically, τ_{D} is related to the fiber-to-target distance (R) and the speed of laser (v) in the medium (e.g., fluid) of the laser path through the relation given by Equation (1):

τ_{D}=2R/v (1)

A heterodyne beat f_{beat}, which represents a frequency difference between the two optical fields corresponding to the chirped laser LO and the return laser signal RX at any given time t, can be determined using the time delay τ_{D} and the chirp rate (κ) that represents the rate of change in the instantaneous frequency (in units of hertz per second), as follows:

f_{beat}=κ*τ_{D} (2)

Combining the Equations (1) and (2) above, the fiber-to-target distance can be determined using Equation (3) as given below:

R=f_{beat}*v/2κ (3)

FIG. 4B shows a full-waveform distance profile 420 computed using a Fourier transform of the heterodyne beat f_{beat}. The profile shows the magnitude (power) of f_{beat} (dB, in logarithmic scale on the y-axis) at different frequencies which can be converted to distances on the x-axis. The peak power 422 of f_{beat} corresponds to the fiber-to-target distance R. In an example, the fiber-to-target distance estimator 216 can measure the heterodyne beat f_{beat} over time (i.e., the time-varying frequency difference between the chirped laser LO and the return laser signal RX), compute the Fourier transform of the heterodyne beat f_{beat}, and determine the fiber-to-target distance 424 that corresponds to the peak magnitude (power) of f_{beat}.

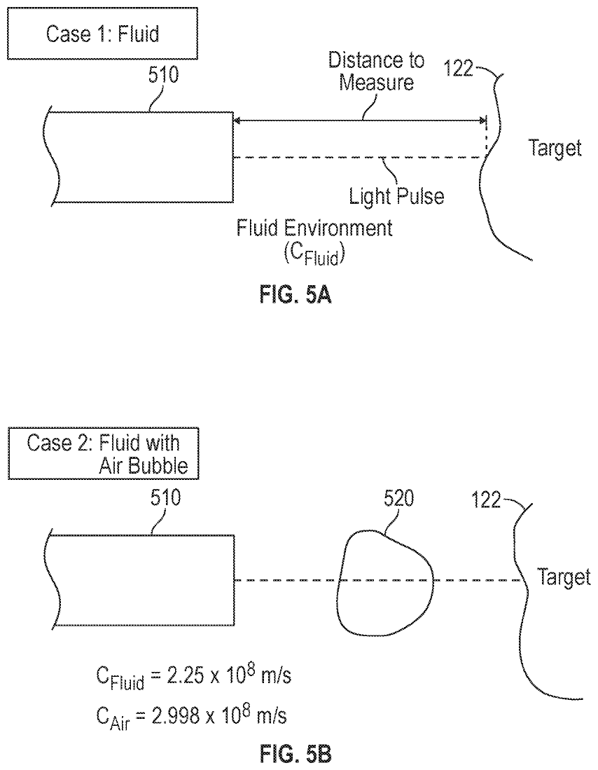

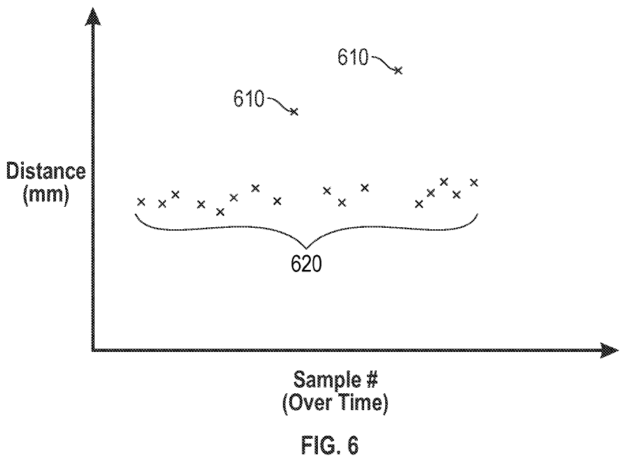

The fiber-to-target distance estimated using the FMCW method as described above can be sensitive to the speed of laser (v) in the medium (e.g., fluid) at the target site. During an endoscopic laser procedure, air bubbles can be generated from the laser energy and fluid irrigation and aspiration. Depending on the type of procedure and target to be treated, tissue debris or calculi fragments may be produced. The air bubbles, tissue debris, calculi fragments, among other particles or objects may cause non-uniformity in the fluid space along the laser path, affect the laser transmission speed (v) and thus interfere with fiber-to-target distance measurement. FIGS. 5A and 5B illustrate examples of how air bubbles interfere with the fiber-to-target distance measurement. FIG. 5A shows a laser signal emitted from a distal end 510 of the laser fiber traveling across a uniform fluid environment free of bubbles or other interfering objects (e.g., tissue debris, calculi fragments) at a speed v_{fluid}, reaching the target structure 122, which reflects at least a portion of the laser signal (the return laser signal) that travels back to the laser fiber tip through the same uniform fluid medium at the speed v_{fluid}. FIG. 5B shows a laser signal, emitted from the distal end 510 of the same laser fiber, traveling across a non-uniform fluid environment filled with air bubbles 520. Because water is denser than air, its refractive index is greater than that of air (~1.3 in water and ~1.0 in air). Compared to the speed of laser in the air cₐᵢᵣ of about 3*10⁸ meters per second (m/sec), the speed of laser in fluid v_{fluid} is approximately 2.25*10⁸ m/sec. Accordingly, when bubbles are present in the light path of the fluid medium, the laser travel time is longer than in bubble-free fluid medium which would be calculated as a greater (than actual) distance when assuming the entire light path is fluid free of bubbles or other interfering objects. This is referred to as an overestimate of fiber-to-target distance. FIG. 6 illustrates an example of overestimate of the fiber-to-target distance due to air bubbles or other interfering objects present in the fluid space of the light path. As illustrated, the fiber-to-target distance can be continuously or periodically measured over time using the optical coherence method as described above. The resulting time series of distance measurements shows overestimates 610 of fiber-to-target distance at those times when air bubbles or other interfering objects are present in the light path between the laser fiber tip and the target. The overestimates 610 are “outliers” greater than other distance measurements 620 done in an bubble-free fluid space.

Referring back to FIG. 2, the distance filter 217 can filter a series of fiber-to-target distance measurements obtained over time, as those shown in FIG. 6, to exclude the outliers, such as the overestimates caused by air bubbles or other interfering objects in the fluid space along the light path. In an example, the distance filter 217 may use a statistical method to identify the outliers. Examples of the statistical methods may include Grubb's test (when testing for a single outlier), Tietjen-Moore test, or Generalized Extreme Studentized Deviate (ESD) test. In an example, a distance threshold or an acceptance range may be determined using an average (or other central tendency metrics) of a plurality of distance measurements within a moving window, and a tolerance margin such as a fraction k of a variance or a standard deviation (SD) of the distance measurements within the moving window. Parameters such as the window length and the fraction k may be adjusted to ensure accurate distance measurements. In a non-limiting example, the moving window has a length of 50 consecutive distance measurements. In a non-limiting example, k takes values between 0.25-0.5.

Each distance measurement produced by the fiber-to-target distance estimator 216 can be tested against the distance threshold or the acceptance range, and identified as either an outlier (e.g., an overestimate) if it exceeds the threshold (e.g., average+0.25*SD) or lies outside the acceptance range (e.g., average±0.25*SD), or as a qualified measurement otherwise. To improve the signal-to-noise ratio (SNR), a refined fiber-to-target distance may be calculated as an average (or other central tendency metrics) of a specified number (e.g., 30-50) of qualified distance measurements. The average and the variance (or SD) of distance measurements may be updated as new distance measurements become available and identified as either qualified or outlier measurement. By excluding the outliers, only the qualified measurements are involved in the update process. This allows for continuous and more accurate and robust fiber-to-target distance measurement.

The controller circuit 218 may be coupled by wired or wireless connections to the feedback analyzer 212. The controller circuit 218 may control the laser system 230 according to one or more control algorithms described herein to control the laser output of the laser source 232. In some examples, the feedback analyzer 212 may continuously monitor the target structure 122, and continuously communicate with the controller circuit 218 to provide feedback control signals to adjust laser output, such as by increasing or decreasing the pulse amplitude, pulse rate, power intensity, duration, frequency, pulse shape, exposure time, among other laser irradiation parameters. The controller circuit 218 may continue maintaining the laser system 230 in a particular state (with a particular output) until a change in feedback is detected. For example, when the target identification circuit 214 detects a different target type or composition based on spectroscopic properties from the spectrometer 213, the controller circuit 218 may adjust the laser output of the laser source 232. In an example, for a renal stone with a hard surface with a first composition and a softer core of a second composition, continuous tissue composition through the target identification allows a first higher laser output to be used to dust the hard surface of a renal stone, and after dusting automatically or upon user confirmation switching to a different lower laser output to ablate the soft core of the stone. As an alternative to the automatic adjustment of laser output, in some examples, the controller circuit 218 may adjust the laser output in a commanded mode, in which case the controller circuit 218 may present to a user (e.g., a surgeon or an endoscopist) current laser output and information about identified target type or composition via a user interface, and recommend the user to adjust the laser output to produce desired therapeutic effect on the target structure 122.

In addition to the target identification information (e.g., target type or composition), the controller circuit 218 may control the laser system 230 to deliver laser energy to the target structure 122 further based on the estimated fiber-to-target distance. For example, if the target structure 122 is identified as an intended treatment structure type (e.g., a specified soft tissue type or a specified calculus type), and if the fiber-to-target distance (d) satisfies a condition (e.g., falling below a threshold dₜₕ or within a specified laser firing range), then the laser pulses may be delivered to the target structure 122. However, if the target structure 122 is not within the laser firing range (e.g., d>dₜₕ), then the controller circuit 218 may produce a control signal to temporarily “lock” the laser source 232, such that no laser pulses are emitted to the target until the target structure 122 is within the laser firing range. The estimated fiber-to-target distance and an indication that the target structure 122 is out of laser firing range (d>dₜₕ) may be presented to the user on a user interface. The user may adjust the optical fiber 234 such as repositioning the distal end of the optical fiber 234 to move closer to the target.

In some examples, the controller circuit 218 may generate a control signal to a robotic device, such as an actuator 260, to robotically adjust the position or the orientation of the distal end of the optical fiber 234 with respect to the target structure 122. For example, the actuator 260 may, in response to the control signal from the controller circuit 218, automatically advance or retract the optical fiber or change an orientation (e.g., an aiming angle) of the distal end of the optical fiber 234 with respect to the target structure 122. In an example, the controller circuit 218 may adjust the position or the orientation of the distal end of the optical fiber based on the identified target type or composition. A desired or optimal distance or range of distance for firing laser at the target may depend on multiple factors including the target type or composition, target location and surrounding anatomy, laser setting, procedure type, or desired tissue effect. As described above, laser output may be adjusted manually or automatically based on target type or composition, such that different portions of the target (e.g., the surface and the core of a calculi structure with respective different compositions) may be treated using different laser outputs. In addition or alternative to the adjustment of laser output, in some examples, the position or the orientation of the distal end of the optical fiber may be adjusted based on target type or composition. In an example of laser lithotripsy, as the target identification circuit 214 continuously analyzes the target type and composition, the controller circuit 218 may control the actuator 260 to advance the distal end of the optical fiber 234 closer to a renal stone target in response to an identification of a hard surface of the target to better dust the stone surface. In response to an identification of a soft core of the stone target, the actuator 260 control the actuator 260 to retract the distal end of the optical fiber 234 further away from the renal stone target.

The user interface 250 may be operatively in communication with the feedback control system 210. The user interface 250 can include a display unit to display information including, for example, surgical site conditions such as images, pressure, or other information sensed by the sensors 220, information generated by the feedback analyzer 212 including the target identification and estimated fiber-to-target distance, and current device settings such as the laser output setting. The display unit can display UI elements including visual elements, alerts, tactile feedback, or any combination thereof. In some examples, the user interface 250 may generate an alert if the fiber-to-target distance exceeds a threshold or a specific range. The alert can be presented in an audible, visible, tactile, or otherwise human-perceptible format. The user interface 250 may include one or more input units to receive user programming of various components of the laser surgical system 200, such as parameter values used for identifying target type or composition, estimating a fiber-to-target distance, and laser output setting. In some examples, the display unit may generate recommendations for adjusting the position or the orientation of the distal end of the optical fiber 234, or for adjusting laser output or other system parameters. A user may use the one or more input units to confirm, reject, or modify any of the recommended adjustments.

FIG. 3 illustrates an example of a feedback-controlled endoscopic laser surgical system 300 with automatic fiber-to-target distance measurement and optical fiber position control. The system 300 can be an example implementation of the laser surgical system 200.

The system 300 may include an endoscope 301, integrated with a feedback control system 310, a laser system comprising a laser source 332 and an optical fiber 334, and a robotic device such as an actuator. The endoscope 301 has a proximal portion and an elongate distal portion configured to be inserted into a surgical site of a patient during an endoscopy procedure. The endoscope 301 may provide visual inspection or treatment of soft (e.g., non-calcified) or hard (e.g., calcified) targets, including but not limited to calculi structures. As illustrated in FIG. 3, the endoscope 301 may include or provide visualization and illumination optics, such as a visualization optical pathway 360 and an illumination optical pathway 350, each of which may extend longitudinally along the elongate body of the endoscope 301. An eyepiece or camera or imaging display may be provided at or coupled to the visualization optical pathway 360 to permit user or machine visualization of a target region at or near a distal end of the endoscope 301. The target region may be illuminated by light 370, such as provided by an illumination light source 324 at a proximal end of the illumination optical pathway 350 and emitted from a distal end of the illumination optical pathway 350. The light source 324 can include, for example, a Xenon lamp, a light-emitting diode (LED), a laser diode, or any combination thereof. In an example, the light source 324 may include two or more light sources that emit light having different illumination characteristics, referred to as illumination modes. In an example, the illumination modes may include a white light illumination mode, or a special light illumination mode such as a narrow band imaging mode, an auto fluorescence imaging mode or an infrared imaging mode. A special light illumination can concentrate and intensify specific wavelengths of light, for example, resulting in a better visualization of tissue or other structures at the surgical site.

The endoscopic laser surgical system 300 may include or be coupled to the laser source 332, which may be an example of the first laser source 106 or the second laser source 116 in FIG. 1, or the laser source 232 in FIG. 2. The laser source 332 may be mechanically and optically connected to the optical fiber 334, which may include a single optical fiber or a bundle of optical fibers. The optical fiber 334, which is an embodiment of the first optical pathway 108 or the second optical pathway 118 in FIG. 1, or the optical fiber 234 in FIG. 2, may be introduced via a proximal access port of the endoscope 301, and extend within a working channel or other longitudinal passage or lumen of the endoscope 301 or similar instrument.

In some examples, the laser source 332 may include a first laser source (such as the first laser source 106 shown in FIG. 1) configured to generate therapeutic laser pulses (also referred to as the treatment beam) for surgically removing or sampling tissue or ablating a calculi structure, and a second lase source (such as the second laser source 116 shown in FIG. 1) to generate non-therapeutic laser pulses such as excitation laser pulses used for estimating a fiber-to-target distance. The therapeutic laser pulses and the non-therapeutic laser pulses can be directed to the target through the same or a different optical pathways.

The endoscopic laser surgical system 300 can include a camera or imaging device 325. The camera or imaging device 325 can include an imaging sensor (such as the imaging sensor 222 in FIG. 2) that can generate an imaging signal 365 of the target in response to electromagnetic radiation (e.g., illumination light 370) of the target at or near the surgical site. The camera or imaging device 325 can be a CCD or CMOS camera, or a laser scanning device. As illustrated in FIG. 3, the target structure 122 is within the view of the camera or imaging device 325, such that in response to the electromagnetic radiation, the camera or imaging device 325 may collect the signal reflected from target structure 122 and produce an imaging signal 365 of the target structure 122. The imaging signal 365 may be transmitted through the optical pathway 360, or alternatively through the optical fiber 334, to the feedback control system 310 (which is an example of the feedback control system 210). In an example, the optical fiber 334 can concurrently direct the laser pulses (including the chirped laser 383 and the return laser signal 385) and the reflected imaging signal 365. The feedback control system 310 can include a feedback analyzer 312 and a controller circuit 318. In an example, the imaging signal may pass through an optical splitter before reaching the feedback analyzer 312. The feedback analyzer 312, which is an example of the feedback analyzer 212 in FIG. 2, may include a spectrometer that may generate one or more spectroscopic properties from the imaging data. The feedback analyzer 312 may identify the target as one type of tissue or one type of calculi of distinct compositions using the one or more spectroscopic properties, as described above with respect to FIG. 2.

The feedback analyzer 312 may calculate or estimate a fiber-to-target distance between a distal end 336 of the optical fiber 334 and the target structure 122. In an example, the fiber-to-target distance may be estimated using a FMCW method, in which a coherence metric may be derived using a chirped laser 383 emitted from the laser source 332, and a return laser signal 385 in response to a portion of the chirped laser irradiating at the target structure 122, as described above with respect to FIGS. 2 and 4A-4B. The controller circuit 318 may generate a control signal to the laser source 332 to adjust an output setting for the therapeutic laser pulse. The adjustment of the therapeutic laser output setting can be based at least in part on the identified target type or composition. In some examples, the adjustment of the therapeutic laser output setting may further be based on the determined fiber-to-target distance. For example, the controller circuit 318 may temporarily “lock” the laser source 332 to prevent it from firing laser pulse if the estimated fiber-to-target distance exceeds a threshold range.

The controller circuit 318 can additionally or alternatively generate a control signal to a robotic device to adjust a position or an orientation of the distal end 336 of the optical fiber 334. The robotic device, such as the actuator 338, can be coupled to a portion of the optical fiber 334, and can be in electrical communication with the controller circuit 318. In an example, the actuator 338 may be located at or near the distal end of the endoscope 301. The actuator 338 may include one or more of an electromagnetic element, an electrostatic element, a piezoelectric element, or other actuating element such as to actuate or otherwise permit longitudinal or rotational positioning of the distal end 336 of the optical fiber 334 with respect to the working channel or other longitudinal passage of the endoscope 301, or with respect to another reference location for which the endoscope 301 may serve as a frame of reference. Based at least in part on the identified target type or composition, the controller circuit 318 can activate the actuator 338 to adjust the position or the orientation of a distal end 336 of the optical fiber 334, such as adjusting the longitudinal position by advancing or retracting the distal end 336 to respectively increase or decrease a distance to the target structure 122, or adjusting the rotational position by steering the distal end 336 to increase or decrease the aiming angle with respect to the target structure 122. Such adjustment of the position or the orientation of a distal end of the optical fiber can improve efficacy of laser treatment while preserving laser energy.

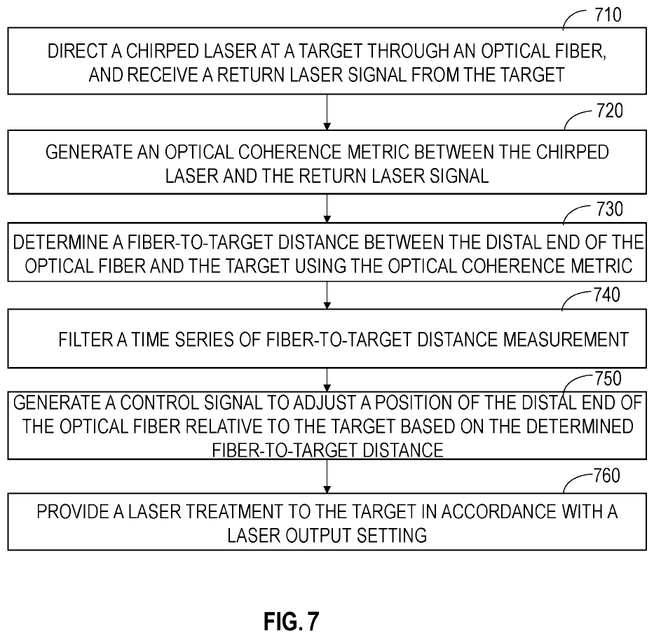

FIG. 7 is a flow chart illustrating an example method 700 of providing feedback-control of a surgical laser system to provide adjustable laser treatment of a target using feedback including fiber-to-target distance measurements. The method 700 may be implement and executed in the laser surgical system 200 shown in FIG. 2, or the feedback-controlled endoscopic laser surgical system 300 shown in FIG. 3, and used for laser lithotripsy of renal stones, bezoars, gallstone, among other calculi structures, or for laser incision or vaporization of soft tissue, such as during an endoscopy procedure. Although the processes of the method 800 are drawn in one flow chart, they are not required to be performed in a particular order. In various examples, some of the processes can be performed in a different order than that illustrated herein.

At 710, a chirped laser may be directed at a target structure, and a return laser signal may be received in response to the chirped laser irradiating at the target. The chirped laser may be emitted from a laser source through an optical fiber of a laser system, such as the laser surgical system 200 or the feedback-controlled endoscopic laser surgical system 300. The chirped laser, also known as a frequency-swept laser, has a time-varying instantaneous frequency. The chirped laser may be split into a first portion that travels through the optical fiber to the target structure, and a second portion being kept local and does not travel to the target structure. A return laser signal can be detected in response to the first portion of the chirped laser irradiating at the target. The first portion of the chirped laser is also referred to as chirped laser excitation. In an example, the chirped laser is a linear chirp, such that the optical frequency of the chirped laser is a linear ramp function of time. As described above in an example with respect to FIG. 4A, the return laser signal may have similar time-frequency profile (e.g., a linear ramp frequency over time) as the chirped laser directed to the target, except that the return laser is substantially a time-delayed version of the excitation chirped laser, where the time delay related to the roundtrip travel time between the distal end of the laser fiber and the target structure.

At 720, an optical coherence metric between the chirped laser excitation and the return laser signal may be generated. The optical coherence metric is correlated to the chirped laser's roundtrip travel time between the distal end of the optical fiber and the target structure. At 730, a distance between the distal end of the optical fiber and the target structure (the “fiber-to-target distance”) may be determined using the coherence metric. Such optical coherence-based distance measurement is also known as frequency modulated continuous wave (FMCW) method. As describe above with respect to FIG. 4B, the FMCW method involves detecting a heterodyne beat f_{beat} representing a frequency difference between the two optical fields corresponding to the chirped laser excitation and the return laser signal at any given time, and identifying peak magnitude (power) of f_{beat} in a frequency domain (such as a Fourier transform of the heterodyne beat f_{beat}). The fiber-to-target distance may be determined as the frequency converted distance corresponding to the peak magnitude (power) of f_{beat}.

At 740, a time series of fiber-to-target distance measurements obtained during continuous or periodic distance using the optical coherence as described above may be filtered, such as using the distance filter 217 shown in FIG. 2. One or more outlier measurements may be identified from the fiber-to-target distance measurements. The outliers, as described above with respect to FIG. 5B, may include overestimates caused by air bubbles generated from the laser energy and fluid irrigation and aspiration, tissue debris, calculi fragments, among other particles or objects. The air bubbles and interfering objects may cause non-uniformity in the fluid space and affect the laser transmission speed, which may lead to overestimates of the fiber-to-target distance. A statistical method may be used to identify the outlier measurements, and filter them out of the data when determining the fiber-to-target distance. Examples of the statistical methods may include Grubb's test (when testing for a single outlier), Tietjen-Moore test, or Generalized Extreme Studentized Deviate (ESD) test. In an example, the outlier measurements may be identified based on an average and a variance or standard deviation (SD) of the plurality of fiber-to-target distance measurements. In an example, a distance threshold or an acceptance range may be determined based on a moving-average (mean, or other central tendency) of a plurality of distance measurements in a moving window, and a tolerance margin such as a fraction k of the SD of the distance measurements within the moving window. Each new distance measurement can be tested against the distance threshold or the acceptance range and identified as either an outlier if it exceeds the threshold or lies outside the acceptance range, or as a qualified measurement otherwise. A refined fiber-to-target distance may be calculated as an average of a specified number of qualified distance measurements.

At 750, a control signal may be generated to the surgical laser system to adjust a position or an orientation of the distal end of the optical fiber relative to the target based at least in part on the determined fiber-to-target distance. The adjustment may be carried out using a robotic device, such as the actuator 260 shown in FIG. 2 that can robotically advance or retract the optical fiber, or change an aiming angle of the distal end of the optical fiber with respect to the target structure. For example, if the fiber-to-target measurement exceeds a distance threshold, the optical fiber may be robotically manipulated to advance the distal end closer to the target. Additionally or alternatively, the position or the orientation of the distal end of the optical fiber relative to the target may be adjusted based on target type or composition, which can be identified based at least in part on the spectroscopic property of the target, such as using the target identification circuit 214 as described above with respect to FIG. 2.

At 760, a laser treatment may be provided to the target structure in accordance with a laser output setting. The laser output setting includes one or more laser output parameters such as pulse amplitude, pulse rate, power intensity, duration, frequency, pulse shape, exposure time, among other laser irradiation parameters. The laser output setting may be adjusted based on target type or composition, which can be identified based at least in part on the spectroscopic property as described above with respect to FIG. 2. Additionally or alternatively, the laser output setting may be adjusted based on the fiber-to-target distance measurement. For example, if the target structure is identified as an intended treatment target type (e.g., a particular soft tissue or calculi structure), and if the fiber-to-target distance satisfies a specific condition (e.g., falling below a distance threshold or within a specified laser firing range), then the laser pulses may be delivered to the target structure. If the target structure is not within the range of the laser, then the laser source may be temporarily “locked” such that no laser pulses are emitted to the target until the target structure is within the laser firing range. In some examples, the laser output setting, the identification of the target type or composition, and the fiber-to-target distance measurement may be presented to a user on a user interface. The user may accept, reject, or modify the laser output setting such as via a user interface before it is applied to the laser system to initiate or adjust the laser treatment to the target.

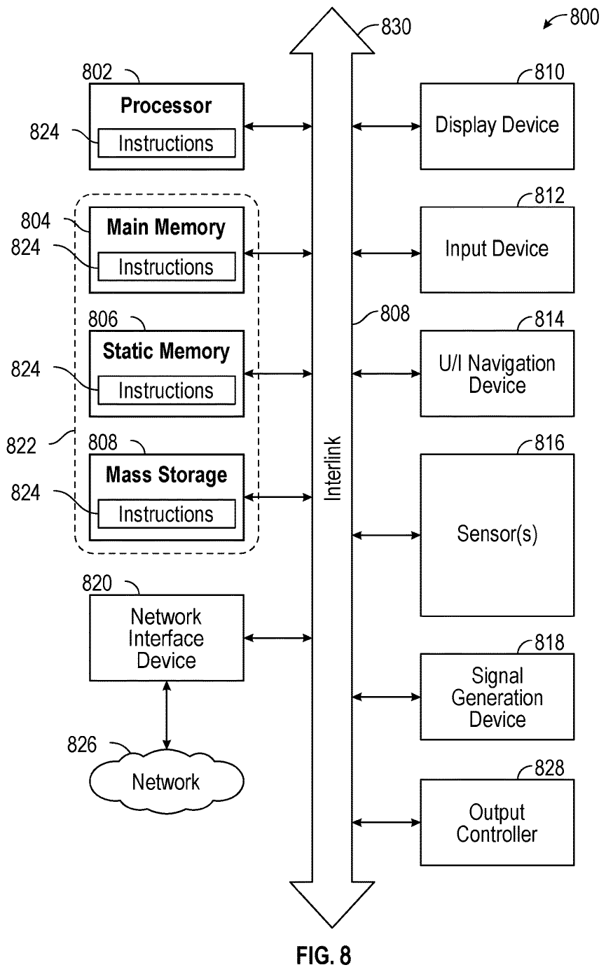

FIG. 8 illustrates generally a block diagram of an example machine 800 upon which any one or more of the techniques (e.g., methodologies) discussed herein may perform. Portions of this description may apply to the computing framework of various portions of laser surgical system 200 or the endoscopic laser surgical system 300.

In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively connected to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810 (e.g., a raster display, vector display, holographic display, etc.), an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensors. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine readable media.

While the machine-readable medium 822 is illustrated as a single medium, the term “machine readable medium” may include a single medium or multiple media (e.g., a centralized or distributed database, and/or associated caches and servers) configured to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine-readable medium with a plurality of particles having invariant (e.g., rest) mass. Accordingly, massed machine-readable media are not transitory propagating signals. Specific examples of massed machine-readable media may include non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 824 may further be transmitted or received over a communication network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communication network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

### ADDITIONAL NOTES

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventors also contemplates examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment.

Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A surgical laser system, comprising:
a laser system configured to generate and deliver laser pulses to a target in an anatomical environment of a patient via an optical fiber; and
a controller circuit, including a feedback analyzer circuit configured to:
in response to a chirped laser emitted from the surgical laser system irradiating at the target, receive a return laser signal from the target;
generate an optical coherence metric using at least a portion of the chirped laser and the return laser signal, the optical coherence metric correlated to roundtrip laser travel time between a distal end of the optical fiber and the target; and
determine a fiber-to-target distance between the distal end of the optical fiber and the target using the optical coherence metric,
wherein the controller circuit is configured to generate a control signal to adjust a position or an orientation of the distal end of the optical fiber relative to the target based at least in part on the determined fiber-to-target distance.

2. The surgical laser system of claim 1, wherein the feedback analyzer circuit is configured to determine the fiber-to-target distance further based on a chirp rate of the chirped laser.

3. The surgical laser system of claim 1, wherein the controller circuit is configured to adjust a surgical laser output setting of the surgical laser system based at least in part on the determined fiber-to-target distance.

4. The surgical laser system of claim 1, further comprising a light source configured to direct an electromagnetic radiation at the target,
wherein the feedback analyzer circuit is configured to:
detect a reflected imaging signal from the target in response to the electromagnetic radiation at the target;
determine a spectroscopic property of the target from the reflected imaging signal; and
identify a target type or composition based at least in part on the determined spectroscopic property of the target.

5. The surgical laser system of claim 4, wherein the controller circuit is configured to generate the control signal to adjust the position or the orientation of the distal end of the optical fiber relative to the target further based on the identified target type or composition.

6. The surgical laser system of claim 4, wherein the controller circuit is configured to adjust a surgical laser output setting of the surgical laser system based at least in part on the identified target type or composition.

7. The surgical laser system of claim 4, wherein the optical fiber is configured to concurrently direct the laser pulses and the reflected imaging signal.

8. The surgical laser system of claim 1, wherein the controller circuit is configured to provide the control signal to a robotic device coupled to the optical fiber to robotically adjust the position or the orientation of distal end of the optical fiber relative to the target.

9. The surgical laser system of claim 1, comprising a user interface configured to present to a user the determined fiber-to-target distance and a recommendation to adjust the position or the orientation of the distal end of the optical fiber relative to the target.

10. The surgical laser system of claim 1, wherein the target includes a tissue target,
wherein the surgical laser system is configured to generate and deliver the laser pulses to treat the tissue target.

11. The surgical laser system of claim 1, wherein the target includes a calculi target,
wherein the surgical laser system is configured to generate and deliver the laser pulses to ablate or fragment the calculi target.

12. The surgical laser system of claim 1, comprising an endoscope including or coupled to the surgical laser system, the endoscope including a longitudinal passage for passing the optical fiber.

13. The surgical laser system of claim 1, wherein the feedback analyzer circuit is further configured to:
identify one or more outlier measurements from a plurality of fiber-to-target distance measurements generated over time;
filter the plurality of fiber-to-target distance measurements to exclude the identified one or more outlier measurements; and
determine the fiber-to-target distance using the filtered plurality of fiber-to-target distance measurements.

14. The surgical laser system of claim 13, wherein the feedback analyzer circuit is configured to identify the one or more outlier measurements based on an average and a variance of the plurality of fiber-to-target distance measurements.

15.-25. (canceled)
